(19) United States
(12) Patent Application Publication
(10) Pub. No.: US 2023/0050810 A1
(43) Pub. Date: Feb. 16, 2023

(54) Manufacturing Systems For Biopharmaceutical Products

(71) Applicant: Fisher-Rosemount Systems, Inc., Round Rock, TX (US)

(72) Inventors: John MILLER, Los Angeles, CA (US); Suraj PAI, Glendale, CA (US); Sudheesh NARAYANAN, Bangalore (IN)

(21) Appl. No.: 17/951,552

(22) Filed: Sep. 23, 2022

Related U.S. Application Data
(63) Continuation of application No. 16/930,434, filed on Jul. 16, 2020, now abandoned.
Application No. 16/930,434 is a continuation-in-part of application No. 16/407,288, filed on May 9, 2019, now abandoned.
(60) Provisional application No. 62/668,837, filed on May 9, 2018.

(51) Int. Cl. G16H 70/40; G05B 19/042

(52) U.S. Cl. CPC G16H 70/40; G05B 19/0426

(57) ABSTRACT

A manufacturing system for a biopharmaceutical product includes first and second sets of biopharmaceutical manufacturing equipment located at a first and second enterprise sites, and memory configured to store an enterprise configuration and a process specification. The enterprise configuration includes records of one or more equipment parameters of the multiple pieces of equipment of the first and second sets of biopharmaceutical manufacturing equipment. At least one processor is configured to execute instructions determine whether at least one of the first enterprise site and the second enterprise site is capable of manufacturing the biopharmaceutical product, transmit a generated set of instructions to the determined at least one of the first enterprise site and the second enterprise site, and operate the multiple pieces of equipment at the determined at least one of the first enterprise site and the second enterprise site to manufacture the biopharmaceutical product.

### CROSS-REFERENCE TO RELATED APPLICATIONS

This application is a continuation of U.S. application Ser. No. 16/930,434 filed Jul. 16, 2020, which is a continuation-in-part of U.S. application Ser. No. 16/407,288 filed on May 9, 2019, which claims priority to U.S. Provisional Application 62/668,837 filed on May 9, 2018. The entire disclosures of each of the above applications are incorporated herein by reference.

### FIELD

The invention generally relates to manufacturing systems for biopharmaceutical products.

### BACKGROUND

The field of biopharmaceutical products includes products such as monoclonal antibodies, proteins, enzymes, vaccines, hormones, immunomodulators, growth factors, coagulation factors, thrombolytics, cell and gene therapies, and many more. The development and manufacturing of these products does not involve simple, straightforward step-by-step procedures. Rather, the processes required to develop and ultimately manufacture these products involve condition-dependent biological and chemical transformations, living organisms and manipulation of structurally complex molecules. Each production step involves not only material inputs and transformations, but also process conditions such as temperature, pressure, incubation time, pH, etc. Everything from materials, equipment and environment can affect these process conditions.

The development of a recipe suitable for manufacture is the culmination of a long evolution called the drug development life cycle. First, new molecules are discovered in a lab, where experiments are performed at a very small, petri dish-size scale. Over the next seven to ten years, a drug is developed in phases, with each phase requiring a more well-defined and tailored process to manufacture the drug at a new scale to support the clinical trial and commercial production process. The process to make 10 liters of product for Phase 2 clinical trials is very different than making 10,000 liters for commercial scale production, so the process is scaled up multiple times throughout the drug development cycle. Scaling at each phase of the drug development process requires extensive experimentation, in order to ensure optimization of the output, and that the output's quality, safety and efficacy can be verified. This is one reason why the drug development process is a massive effort, taking ten years or more.

Typically, groups like drug development and/or process development focus on increasing the quantity/scale of how much product can be produced in a single batch. These groups must work across global sites to characterize the attributes and optimal conditions to manufacture newly discovered products. Sites may be located all over the globe, and each site may have different machinery and systems, necessitating site-specific development of the recipe. Thus, over time, the result is that many versions and iterations of recipes, at different scales and for different production locations, are created and stored.

The entire manufacturing process determines the quality of a biopharmaceutical product. Very minor changes may affect composition, and therefore the quality, safety, and efficacy of a drug. Impurities and contaminants may be introduced by process conditions. In other words, the process itself determines the qualities of the resulting product. Thus, every detail of the process must be controlled. Process control is a requirement under this country's regulatory regimes. The Food, Drug & Cosmetics Act § 501(a)(2)(B) dictates that “A drug shall be deemed to be adulterated if the methods used in, or the facilities or controls used for, its manufacture, processing, packaging, or holding do not conform to or are not operated or administered in conformity with current good manufacturing practice to assure that such drug meets the requirements of this Act as to safety and has the identity and strength, and meets the quality and purity characteristics, which it purports or is represented to possess.” The Act's implementing regulations require written procedures for production and process control, which must be followed in the execution and documented at the time of performance. Deviations from these written procedures must be recorded and justified. Written procedures should also describe in-process controls, tests, and examinations to be conducted on appropriate samples of in-process materials of each batch. Such written procedures serve to validate the performance of the manufacturing process.

In the current state of the art, written procedures for biopharmaceutical production are created, stored, and propagated using enterprise document management systems. These written procedures, often referred to as “recipes” or “process specifications,” are the results of long-term, enterprise-wide efforts spanning the drug development life cycle. The various iterations of a recipe, in addition to the insights, institutional knowledge, and experimental data relating to any given procedure, are typically captured in numbered versions of Microsoft, Google, or other word and spreadsheet processing documents and/or enterprise application software, often in an ad-hoc fashion.

When it becomes time for regulatory authorities to review the written procedures for manufacture and process control, painstaking documentation is required. Recipes are often drafted from scratch by reviewing and consolidating information stored in Word, Google, Excel, and other documents, as well as enterprise document management systems. Written procedures for manufacturing execution are prepared at individual sites, each of which has unique machinery, equipment, systems and/or staff. Some sites may be external to the organization, otherwise known as third-party contract development and manufacturing organizations (CDMOs). Because regulations require process control, including documentation and validation at the time of execution, a controlled, secure system to facilitate recipe documentation and transmittal for execution is needed.

### SUMMARY

This section provides a general summary of the disclosure, and is not a comprehensive disclosure of its full scope or all of its features.

According to one aspect of the present disclosure, a manufacturing system for a biopharmaceutical product includes a first set of biopharmaceutical manufacturing equipment located at a first enterprise site at a first physical location, the first set of biopharmaceutical manufacturing equipment including multiple pieces of equipment each having one or more equipment parameters representing an operating capability of the piece of equipment, a second set of biopharmaceutical manufacturing equipment located at a second enterprise site at a second physical location, the second set of biopharmaceutical manufacturing equipment including multiple pieces of equipment each having one or more equipment parameters representing an operating capability of the piece of equipment, and memory configured to store an enterprise configuration and a process specification. The enterprise configuration includes records of the one or more equipment parameters of the multiple pieces of equipment of the first set of biopharmaceutical manufacturing equipment and the one or more equipment parameters of the multiple pieces of equipment of the second set of biopharmaceutical manufacturing equipment, and the process specification including a sequence of steps to manufacture the biopharmaceutical product and one or more step parameters corresponding to each of the steps, each of the one or more step parameters representing a physical attribute related to controlling a process of manufacturing the biopharmaceutical product. The system includes at least one processor configured to execute computer-executable instructions, wherein the instructions include determining, based on the one or more step parameters for manufacturing the biopharmaceutical product and the one or more equipment parameters of the multiple pieces of equipment at each of the first enterprise site and the second enterprise site, whether at least one of the first enterprise site and the second enterprise site is capable of manufacturing the biopharmaceutical product. In response to determining that at least one of the first enterprise site and the second enterprise site is capable of manufacturing the biopharmaceutical product, the instructions include generating a set of instructions that are executable by the multiple pieces of equipment of the determined at least one of the first enterprise site and the second enterprise site, the set of instructions including one or more process control parameters that, when the set of instructions is executed by the multiple pieces of equipment of the determined at least one of the first enterprise site and the second enterprise site, control operation of the multiple pieces of equipment at the determined at least one of the first enterprise site and the second enterprise site. The instructions include transmitting the set of instructions to the determined at least one of the first enterprise site and the second enterprise site, and operating the multiple pieces of equipment at the determined at least one of the first enterprise site and the second enterprise site according to the transmitted set of instructions, to manufacture the biopharmaceutical product.

According to another aspect of the present disclosure, a method for automated manufacturing of a biopharmaceutical product includes obtaining an enterprise configuration including records of one or more equipment parameters of multiple pieces of equipment of a first set of biopharmaceutical manufacturing equipment located at a first enterprise site at a first physical location, and one or more equipment parameters of multiple pieces of equipment of a second set of biopharmaceutical manufacturing equipment located at a second enterprise site at a second physical location, obtaining a process specification including a sequence of steps to manufacture the biopharmaceutical product and one or more step parameters corresponding to each of the steps, each of the one or more step parameters representing a physical attribute related to controlling a process of manufacturing the biopharmaceutical product, and determining, based on the one or more step parameters for manufacturing the biopharmaceutical product and the one or more equipment parameters of the multiple pieces of equipment at each of the first enterprise site and the second enterprise site, whether at least one of the first enterprise site and the second enterprise site is capable of manufacturing the biopharmaceutical product. In response to determining that at least one of the first enterprise site and the second enterprise site is capable of manufacturing the biopharmaceutical product, the method includes generating a set of instructions that are executable by the multiple pieces of equipment of the determined at least one of the first enterprise site and the second enterprise site, the set of instructions including one or more process control parameters that, when the set of instructions is executed by the multiple pieces of equipment of the determined at least one of the first enterprise site and the second enterprise site, control operation of the multiple pieces of equipment at the determined at least one of the first enterprise site and the second enterprise site. The method includes transmitting the set of instructions to the determined at least one of the first enterprise site and the second enterprise site, and operating the multiple pieces of equipment at the determined at least one of the first enterprise site and the second enterprise site according to the transmitted set of instructions, to manufacture the biopharmaceutical product.

Further aspects and areas of applicability will become apparent from the description provided herein. It should be understood that various aspects of this disclosure may be implemented individually or in combination with one or more other aspects. It should also be understood that the description and specific examples herein are intended for purposes of illustration only and are not intended to limit the scope of the present disclosure.

### BRIEF DESCRIPTION OF THE DRAWINGS

FIG. 1 is a block diagram showing an environment in which embodiments of the invention may be implemented.

FIG. 2 is a block diagram showing a process for storing, calculating, and transmitting a biopharmaceutical process specification.

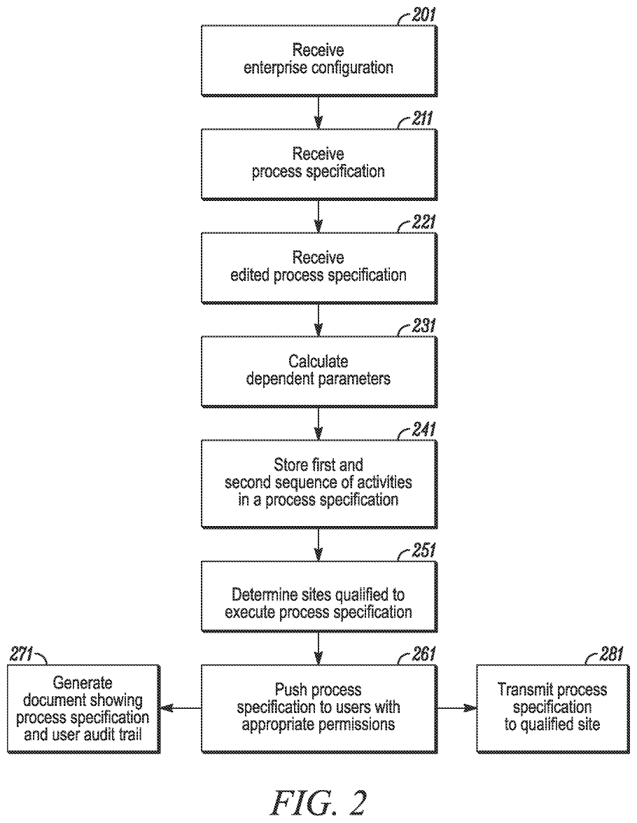

FIG. 3 is a block diagram showing the components of an enterprise configuration.

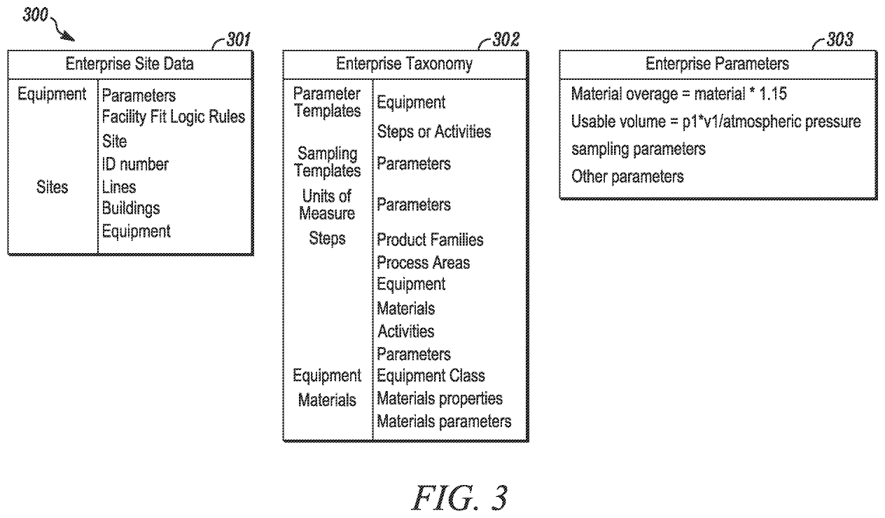

FIG. 4 is a block diagram showing the components of a process specification.

FIG. 5 is an exemplary display of a process specification canvas.

FIG. 6 is an exemplary display of a step menu.

FIG. 7 is an exemplary display of a step menu with configurable fields.

FIG. 8 is an exemplary display of an activity menu with configurable parameters.

FIG. 9 is an exemplary display of a calculation editing screen.

FIG. 10 is an exemplary display of an enterprise materials configuration screen.

FIG. 11 is an exemplary display of an equipment parameter screen.

FIG. 12 is an exemplary display of enterprise location configuration screen.

FIG. 13 is a block diagram showing a sub-process for determining sites qualified for carrying out a process specification.

FIG. 14 is a block diagram showing a process for optimizing parameters of a process specification.

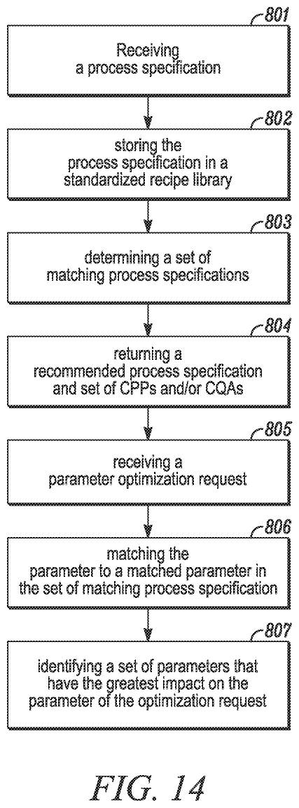

FIG. 15 is an exemplary display of a process specification comparison screen.

### DETAILED DESCRIPTION

“Process specifications,” also known as “recipes,” are a way to define manufacturing process standards that are proposed and justified by manufacturers and reviewed by the regulatory authorities as conditions of approval for commercial use. Process specifications are the outcome of the drug development process. In certain regulatory regimes, they are legally binding criteria. Even in the absence of governmental review, process specifications are an important part of Current Good Manufacturing Practices, or CGMP. Process specifications ensure the consistency and quality of the product and help ensure that it is safe and effective over the shelf life of the product. A process specification is comprised of a sequence of “steps,” many of which are associated with “parameters” having acceptance criteria within numerical limits or ranges. Steps may also involve tests or sampling procedures to determine whether the parameters meet the acceptance criteria.

The invention is a development and management system for biopharmaceutical process specifications. The system is configured to receive an enterprise configuration that comprises enterprise site data and parameters and enterprise rules and specifications regarding biopharmaceutical processes. The system maintains said enterprise configuration in a data structure that enables the system's functionalities. The system is configured to receive a process specification made of steps and associated parameters. In particular, the system is configured to receive parameters that express biopharmaceutical functions of and/or relationships with other parameters. For instance, the liquid to solid ratio at the completion of a fermentation step may be expressed as a function of an inoculant concentration parameter during an inoculation step. It is notable that such functions or relationships may represent institutional knowledge built over years of experience, and indeed, the system is configured such that such parameters can be part of the underlying enterprise configuration.

The system is configured to perform automatic calculations on all of the parameters defined in a process specification. This is particularly valuable, for instance, when many parameters are dependent on each other, or when recipes need to be scaled to increase output—whether because development has moved to a new stage in the biopharmaceutical lifecycle, or because an approved recipe is ready to transfer to another facility with different systems or equipment. Furthermore, the system is configured to automatically determine and display the enterprise sites that are qualified to carry out a given process specification.

To users of the system, the interface appears as a canvas that enables the creation and editing of process specifications. The system is configured to maintain control over users, such that only users with appropriate permissions can view or edit process specifications or parts of process specifications, and all user access is secured and tracked. The system is configured to output process specifications and audit trails in a configurable, systematized form to facilitate internal and regulatory review. Therefore, the system underlies recipe execution according to verifiable process specifications, because a verifiable set of process instructions can be securely transferred to a manufacturing site. The process instructions may also include parameters that are essential to safety and control, such as Critical Process Parameters (CPPs) and Critical Quality Attributes (CQAs), and, using Application Programming Interfaces (APIs), may also be transmitted in a form that is readable to manufacturing site automated execution systems.

This system greatly accelerates new biopharmaceutical recipe development in several respects. Biopharmaceutical development proceeds in phases that stretch over long periods of time, but many of the biopharmaceutical process functions remain the same from phase to phase or across similar products. By providing for an underlying enterprise configuration and programmable, automatic calculations, the system reduces repetitive or duplicative process development across phases or similar products. Similarly, by accessing the set of existing process specifications, the system can determine analogous process specifications and analyze their differences, even suggesting initial process specifications and/or specific steps. Second, particularly during early phases of the development cycle, a great deal of experimental data related to testing of various parts of an initial process specification and the resulting variations in output is generated. Although currently captured in various states in paper or electronic lab notebooks, the experimental data can now be retained as it relates to the process specification as a whole. Third, the system uses real-time messaging so that every update and calculation is automatically pushed to all users with the appropriate permissions. Whereas process development previously proceeded sequentially from one process stage to the next in order to maintain control over information and updates created at each stage, real-time communication using a shared system and process specification data structure enables simultaneous or distributed development. Fourth, because all users use the same system, permissions and audit trails can be implemented. Therefore, the system complies with data compliance regulations and, by enabling the production of audit trails, supports, and accelerates the process of complying with regulatory review. Analogously, the storage of process specifications in a single and secure system accelerates the process of technology transfer, in which a recipe previously approved for manufacture at a first site is approved by regulatory authorities for manufacture at a second site. Lastly, previously, the determination of whether an enterprise site would be capable of carrying out an approved process required transfer to the site for hypothetical site-specific versioning of the process. Enterprises would need to make these determinations before contracting with third party contract manufacturing organizations or internal sites. By providing for automatic determination and display of enterprise sites that would be qualified to carry out a particular process specification, the system cuts down time-consuming hypothetical technology transfer.

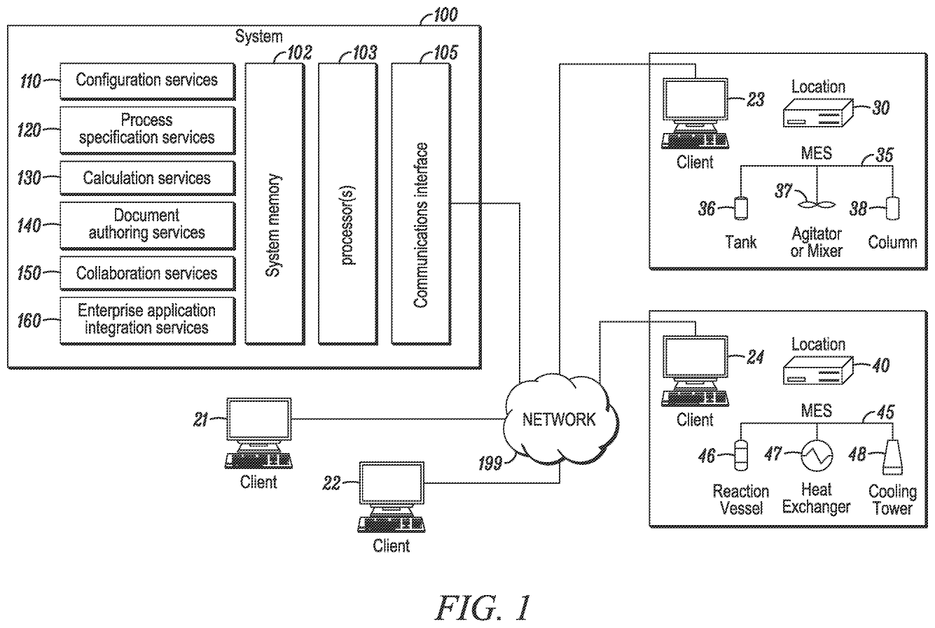

Referring now to the drawings, in which like numerals represent like elements, FIG. 1 is a block diagram showing a networked environment in which the invention may be implemented. System [100] may operate in a networked environment using connections to remote devices through a network such as the Internet. One or more client devices [21-24] may communicate and interact with system [100] through communications interface [105]. Some of the one or more clients may be located at one or more enterprise locations [30; 40]. Particular instruments, such as tanks [36], agitators or mixers [37], columns [38], reaction vessels [46], heat exchangers [47] or cooling towers [48] may communicate with a Manufacturing Execution System (MES) [35, 45] that are linked via a network [199] to the system [100]. Communications interface [105] may allow software and data to be transferred between system [100], a network [199] and client devices [21-24]. Examples of communications interface [105] may include a modem, a network interface (e.g., an Ethernet card), a communications port, etc. System memory [102] can be any memory device configured to store a set of instructions, which may be executed by one or more processors [103]. By way of example, memory [102] may include flash memory devices, Read Only Memory (ROM) devices, Random Access Memory (RAM) devices, as well as hard drives, solid state drives, arrays, etc. The arrangement, connections, and number of components in system [100] is provided for purposes of illustration. System [100] may be implemented on one or more host servers each of which may have their own system memory [102], one or more processors [103] and communications interface [105]. System [100] may even be implemented by the one or more clients. Additional arrangements, number of components, and other modifications may be made, consistent with the present disclosure and the current state of distributed computing technology.

For purposes of clarity and to promote understanding of the complex functionality provided by system [100], and to demonstrate one example of how said system may be implemented, system [100] will sometimes be described in terms of modules that perform particular functions. In particular, FIG. 1 shows modules for configuration services [110], process specification services [120], calculation services [130], document authoring services [140], collaboration services [150], and enterprise application integration services [160], which carry out the functionality of the invention. Terms such as “managers,” “services,” and “engines” are used to describe parts of the system that because of their particular functionality may be described and programmed as separate modules but do not have to be. These modules may run on separate host servers, or they may interact on a single server.

### Enterprise Configuration

The system is configured to receive [201] an enterprise configuration [300] comprising enterprise site data [301], namely, data representing enterprise equipment and associated with sites and equipment parameters; an enterprise taxonomy [302], which is a hierarchy of enterprise data, such as enterprise locations, enterprise equipment, enterprise parameters and enterprise measurement units; and enterprise parameters [303] that are biopharmaceutical functions and formulae common throughout the biopharmaceutical recipes manufactured by the enterprise. The enterprise configuration is the data structure from which the system derives its functionality. The components and fields of enterprise configuration [300] can be stored in JSON format.

Enterprise site data [301] is used by the enterprise to represent the sites and equipment available to the enterprise to manufacture biopharmaceutical recipes. Sites are physical locations at which the enterprise manufactures biopharmaceutical recipes. Some sites house multiple buildings that have different uses, and likewise some buildings house equipment lines that have specific uses. For instance, an enterprise may have a San Diego site with a research building and a manufacturing building, and the manufacturing building may house two lines of fermentation vats and two lines of filtration equipment.

Equipment represents individual items of equipment that are available for use by an enterprise. For instance, an enterprise would understand an individual bioreactor to be an item of equipment. Each equipment is associated with fields representing its unique ID and site, as well as one or more equipment parameters and facility fit logic rules. Equipment parameters would be understood by the enterprise to represent the equipment's properties in relation to the biopharmaceutical process activities that can be performed by said equipment. For instance, equipment may have a flow rate parameter, piping diameter, depth, volume, (each with their own maximum and minimum values), etc. Facility fit logic rules are rules for applying biopharmaceutical process activities to the equipment. An example of this would be a rule stating that the working volume of a particular process step/activity must fall within the minimum and maximum working volume for the equipment, otherwise the equipment is determined to be not qualified to perform the activity. In other words, a facility fit logic rule is a function whose outcome depends on an equipment parameter and a step/activity parameter, which will be discussed further below.

The enterprise configuration is adaptable to the circumstances of the enterprise. For instance, enterprises commonly use proprietary “qualified” maximum and minimum parameters that may differ from manufacturers' published maximum and minimum parameters. The qualified parameters may be applied in particular processes, such as processes previously certified for manufacturing. Thus, the enterprise could choose to create an equipment parameter that includes manufacturer's minimum, qualified minimum, qualified maximum, and manufacturer's maximum. Where multiple parameters share the same structure, the enterprise may further associate these parameters with parameter templates. For instance, any equipment may be associated with a min/max parameter template that includes manufacturer's and qualified minimums and maximums. Parameter templates may be associated with equipment parameters, as well as with parameters pertaining to biopharmaceutical steps/activities, which are described in further detail below. Parameter templates may be an element of the enterprise taxonomy [302].

An enterprise taxonomy [302] represents associations between biopharmaceutical activities performed throughout the enterprise. For instance, certain equipment may be associated with an equipment class. Another example of an enterprise-relevant association that can be represented in enterprise taxonomy [302] is that of biopharmaceutical “activities” with steps, process areas and product families. A biopharmaceutical step is a processing activity that results in chemical or physical change(s) in a material being processed. Examples of steps may include depth filtration, anion exchange chromatography, harvest, production fermentation, perfusion fermentation, inoculum fermentation, etc. Some steps may comprise multiple activities—for instance, the step of “inoculum fermentation” may require media preparation, weighing, inoculation, and transfer conditions. As such, activities are sub-processing activities that describe actions required to create chemical or physical change(s) in a material being processed. As is common in biopharmaceutical process specifications, certain sets of steps can be grouped into “process areas,” i.e., steps that are performed independently from other process areas. Examples include cell cultures, seed trains, buffer preparations, purifications, and media preparations. Along similar lines, certain sets of steps can be associated with product families—for instance, product families such as monoclonal antibodies, or small molecules vs. large molecules, share consistent sets of steps. Thus, in an enterprise taxonomy, steps may be associated with multiple activities, process areas and/or product families.

Biopharmaceutical steps and/or activities will also be associated with fields representing parameters. Parameters would be understood by a biopharmaceutical enterprise to represent attributes that must be monitored or controlled to ensure production quality. For instance, an “Upper/Lower Temperature” parameter may be comprised of the following fields: set point, upper limit and lower limit; a “Pre-Weigh Materials?” parameter may be comprised of a single-select field representing selections “Yes” or “No”; a “Working Volume” parameter may have a minimum, maximum, and set point. Parameters such as “Working Volume” may be so common as to form parameter templates. As previously noted, enterprise taxonomy [302] may include parameter templates, including parameter templates for both equipment and steps/activities.

A parameter may be a function of other, existing parameters. For instance, a minimum working volume of one step/activity may be equal to a percentage of the volume set point of another step/activity. Or, the liquid to solid ratio at the completion of a fermentation activity may be expressed as a function of an inoculant concentration parameter during an inoculation step. In other words, a parameter dependency can be an expression of a biopharmaceutical function. A biopharmaceutical function is any mathematical relationship in which one or more variables or outputs is related to a biopharmaceutical processing step/activity. Parameters often involve complex calculations that were derived from the enterprise's research activities. A first parameter on which a second parameter is dependent may even be defined in sequential relation to the first parameter, such as when a parameter associated with Step A may be dependent on a parameter associated with Step A+1.

Some parameters may also be associated with sampling procedures, which define the processes by which parameters must be measured. For instance, several different methods may be available for sampling and measuring a pH parameter, using different types of instrumentation and techniques. Or, a viable cell density parameter may be measured by visual examination of a small sample, or by a cell density sensor. Each enterprise may have proprietary methods for parameter sampling, which may be included in the enterprise configuration. Some parameters, such as volume parameters, may depend on their sampling parameters, insofar as sampling parameter volume requirements must be added to the volume parameter in order that enough material is available for a process.

Thus, enterprise configuration [300] may comprise a set of enterprise parameters [303] that are functions or formulae common throughout biopharmaceutical recipes manufactured by the enterprise. For instance, certain steps may always require material overages in order to maintain production flow, thus a material overage parameter may be defined as a percentage of material associated with a step. As another example, an equipment usable volume parameter may be calculated by applying Boyle's law to the volume of the particular piece of equipment, and certain types of equipment, such as tanks, may always be associated with a usable volume parameter.

Enterprise taxonomy [302] may also comprise units of measure, which are applied to parameters. Units of measure that are applied in particular recipes may need to be converted when the recipe scales from research to manufacturing, or when transferring the process internationally. Measurement units are crucial when the enterprise spans multiple sites or even countries to ensure that the correct magnitude/scale of the process specification is accurately represented. Any parameter calculation performed by system [100] will include scaling to the appropriate measurement unit.

Enterprise taxonomy [302] may also comprise a listing of materials and associated fields representing their properties and parameters. An enterprise would understand materials to represent the physical inputs and outputs of a step. Materials may be solutions, such as media or buffers; disposable items, such as bags or flasks; active pharmaceutical ingredients; and delivery devices, such as syringes.

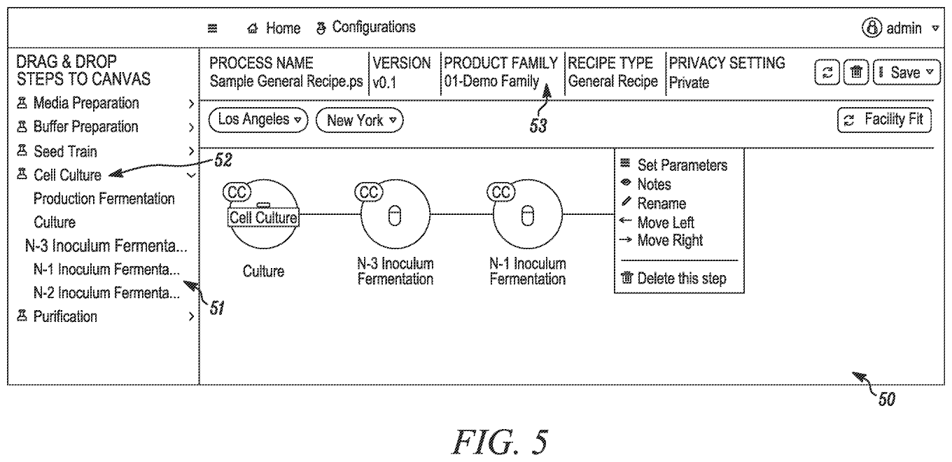

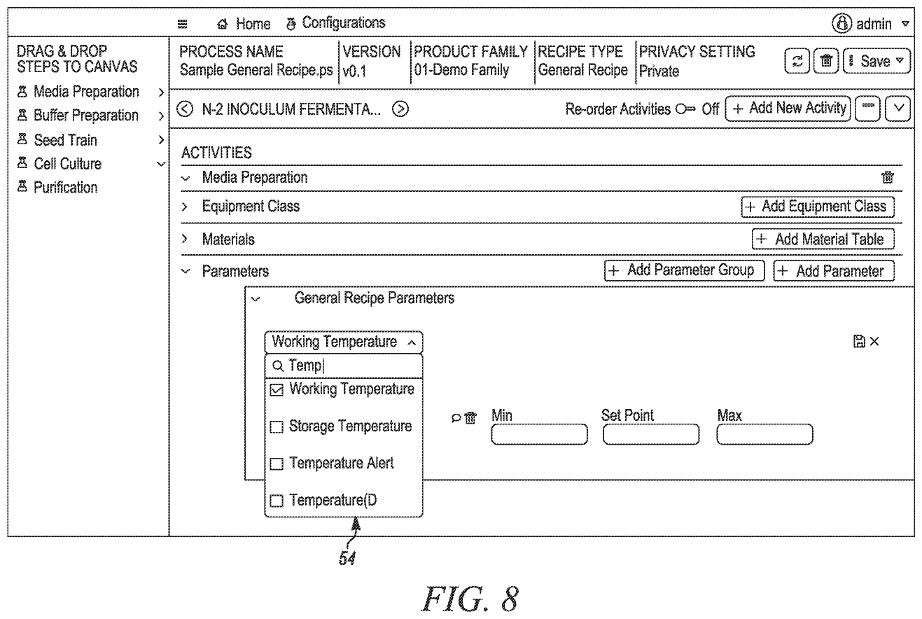

The storage of biopharmaceutical activities, parameter templates and other enterprise data as an enterprise taxonomy [302] facilitates and enhances the development of new recipes. Using hierarchies contained in the enterprise taxonomy, researchers and other users can quickly select and configure steps and activities that would be common to particular product families or process areas. For example, as shown in FIG. 5, process specification canvas [50] shows a menu of steps [51] available under cell culture process area [52]. Product family [53] determines which steps are available to use in the process specification being developed. Another example is shown in FIG. 8, where a set of parameter templates [54] is available by pull-down menu. The enterprise taxonomy leverages enterprise knowledge so that new development does not start from scratch.

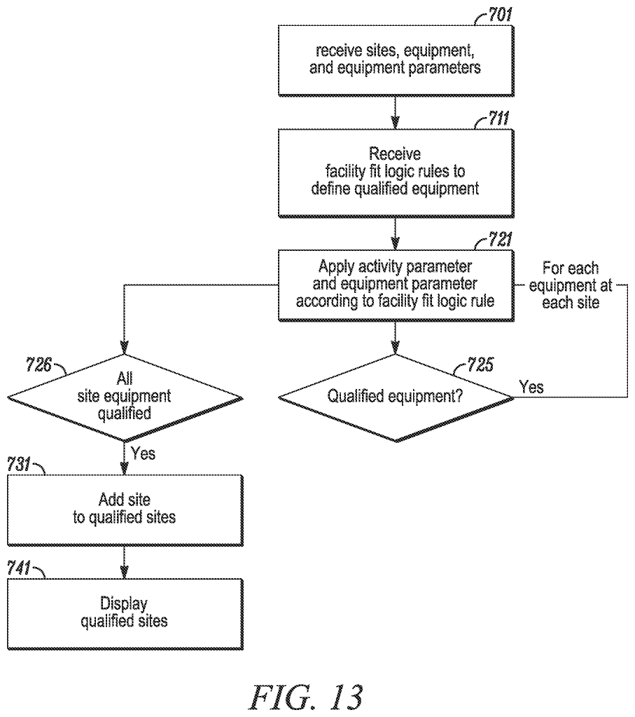

Enterprise configuration [300] is used throughout the system. As will be discussed in more detail below, parameters will be accessed by calculation services [130] in order to perform calculations [231] on dependent parameters and to determine [251] whether a site is qualified to carry out a particular step. See FIG. 13. Elements of the enterprise configuration are also accessed by enterprise application integration services [160] and document authoring services [140] in order to transmit a process specification to a qualifying site or to output a process specification document for review and approval. Enterprise configuration services [110] comprises the implementation of an enterprise configuration database, which may be implemented using any database software, including, but not limited to MongoDB, OrientDB, Couchbase Data or any other noSQL or SQL database solution.

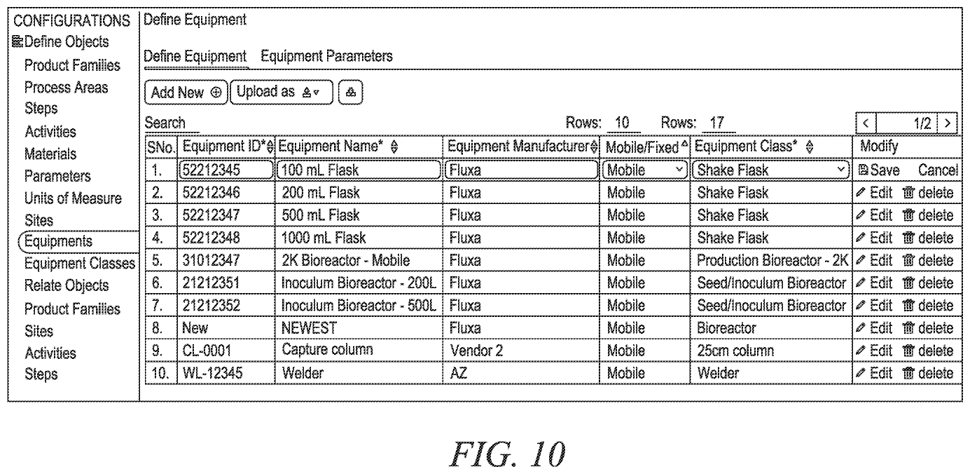

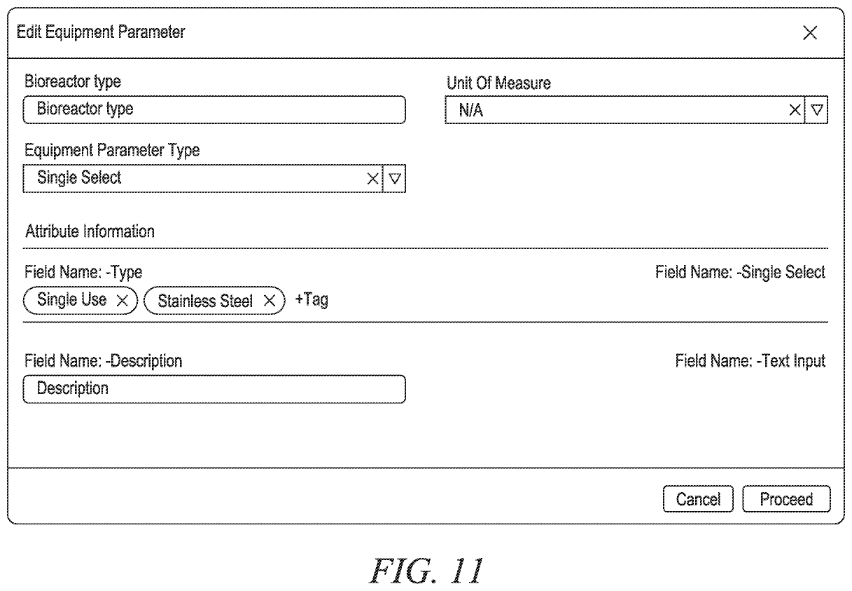

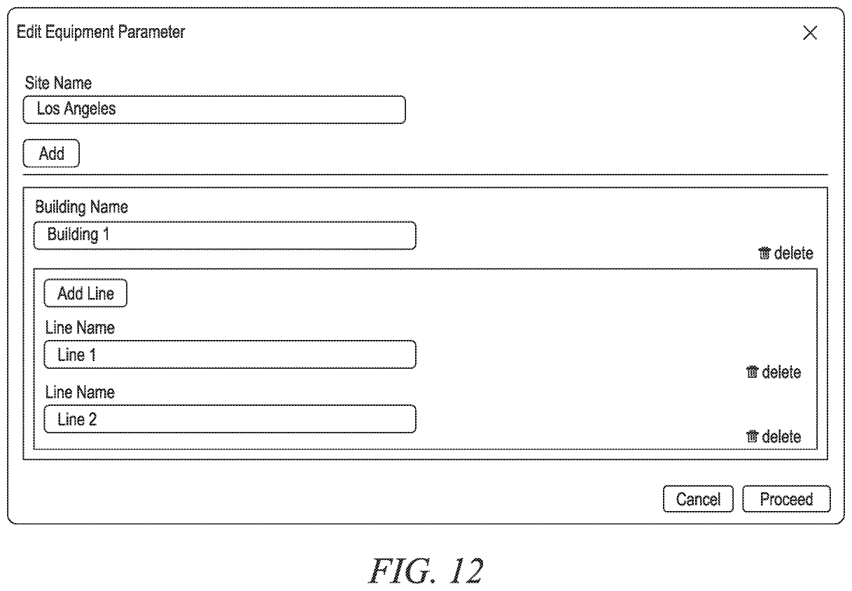

The enterprise configuration is received [201] via communications interface [105]. Enterprise application integration services [160] may implement an API to receive and process existing enterprise data into enterprise configuration [300]. For example, data previously stored in an existing enterprise resource planning tool such as SAP can be imported and processed into an enterprise configuration [300]. Enterprise configuration [300] may also be received from one or more client users [e.g., 21-24] with the appropriate permissions, who enter the required enterprise configuration components through a visual interface implemented in a browser application or other user application. FIG. 10-12 show various examples of displays that could be used to collect enterprise configuration data.

### Process Specifications

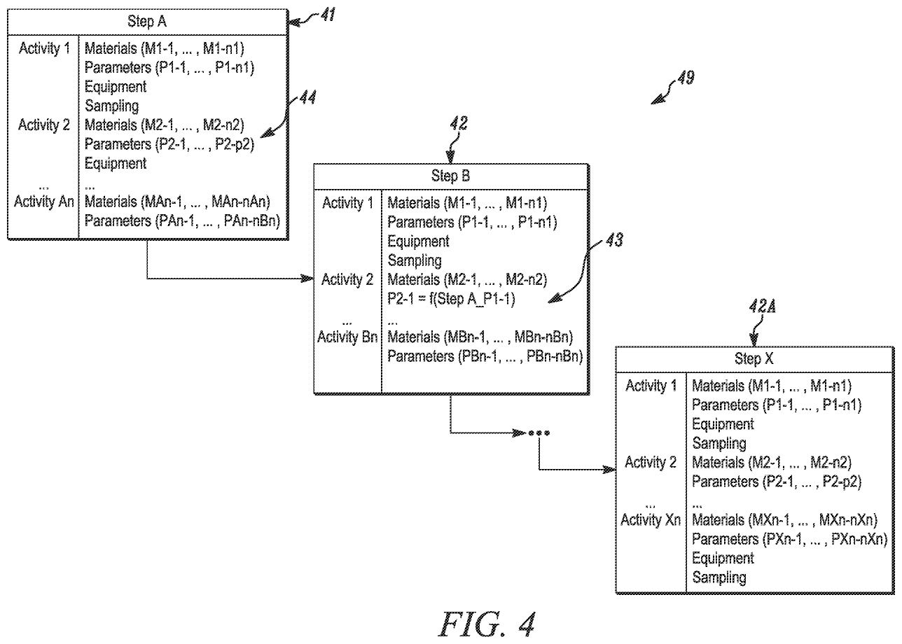

System [100] is configured to create, edit, compare, manage, store, and transmit process specifications. A process specification may be associated with metadata such as the type of molecule, chemical or biological structure, and phase of development. A process specification is comprised of a sequence of steps, each of which may be comprised of a sequence of activities. As previously detailed, each step [41, 42, 42A] or activity represents a processing activity that results in chemical or physical change(s) in a material being processed, may belong to a process area, and may be associated with parameters [43, 44], which represent attributes that must be monitored or controlled to ensure production quality. As shown in FIG. 4, a process specification [49] may include various steps [41, 42, 42A]. Step A [41] may include various activities, each in including materials, parameters [44], and equipment and a parameter [43] of Activity 2 of Step B [42] may be defined as a function of a parameter [44] of Activity 1 of Step A [41]. This function may represent a biopharmaceutical relationship between the steps/activities. Some parameters [43, 44] may also be associated with sampling parameters, which define the processes by which parameters must be measured. Additional informational metadata may be associated with steps, activities and/or parameters, such as information from stored lab experiment data, user comments regarding version changes, process specification data, and manufacturing execution data.

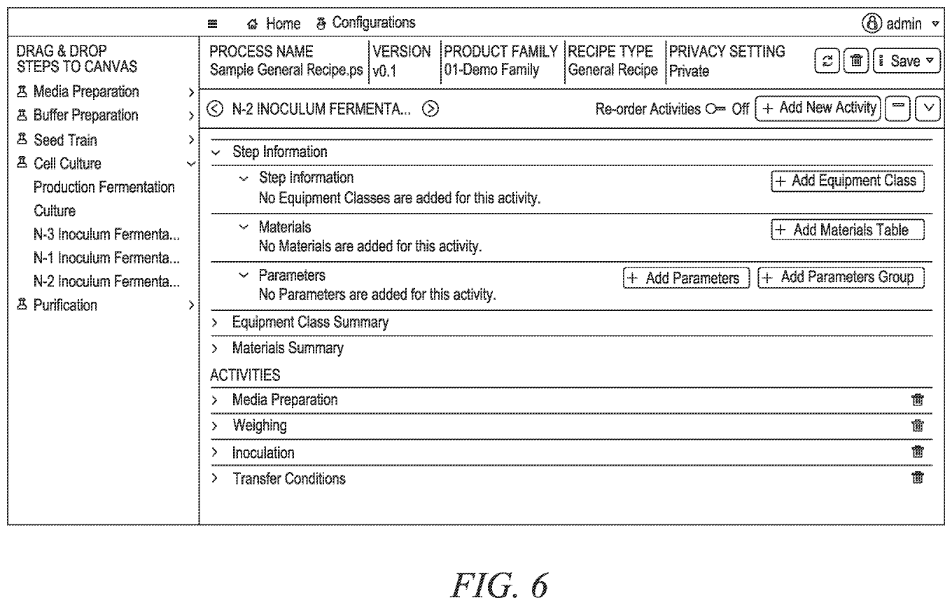

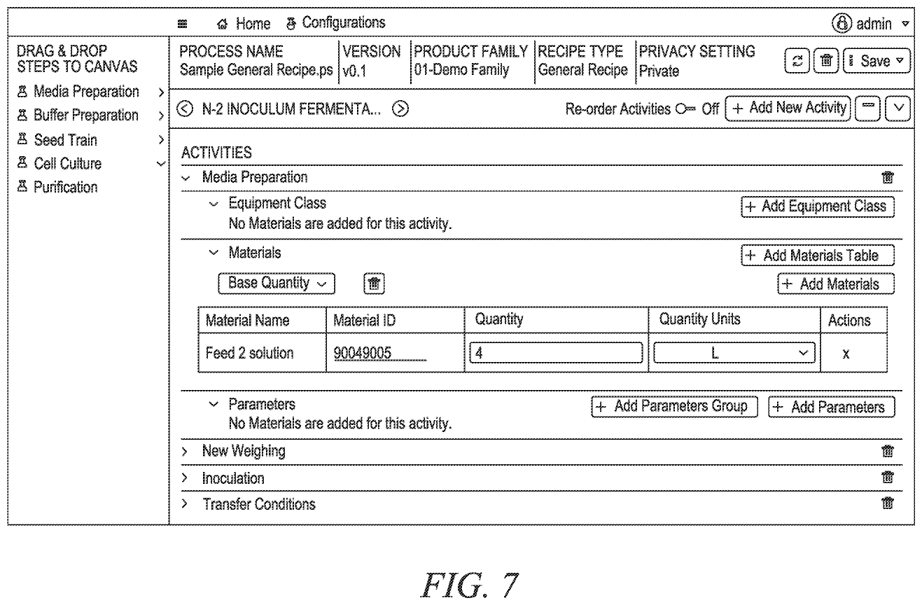

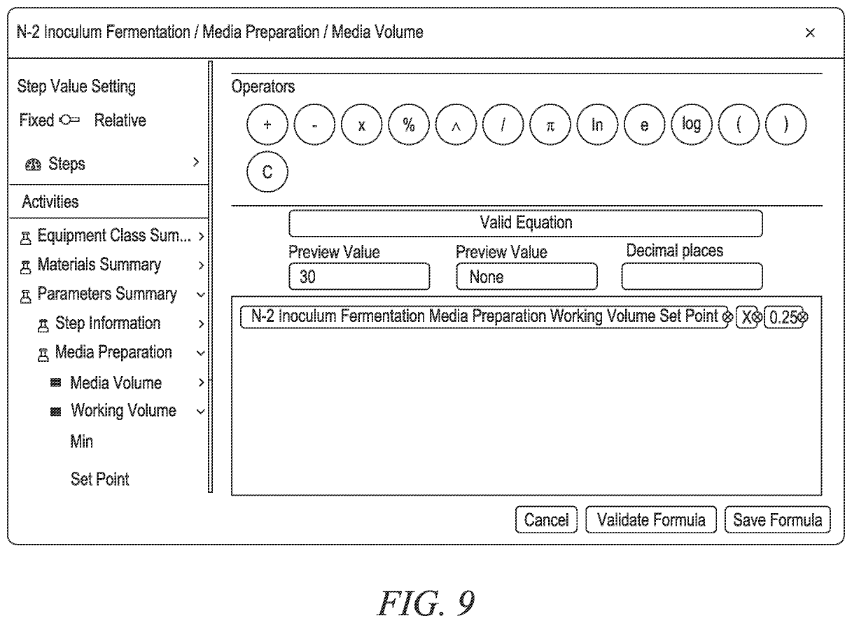

Based on enterprise configuration [300], system [100] is configured to provide a visual user interface for displaying a process specification canvas [50], which facilitates the creation, editing, management, storage, and transmittal of process specifications. An example of an editing interface is shown in FIG. 5, in which steps [51] are represented by icons that can be dragged onto process specification canvas [50] to visualize a process specification. Pursuant to enterprise taxonomy [302], a step may be associated with equipment classes, materials, parameters, activities, etc. Such fields associated with a step may be presented in a step menu as shown in FIG. 6. Materials and parameters for each step or activity may be presented for editing as shown in FIG. 7-9.

Process specification canvas [50] may be displayed at a client terminal [21-24] in a client application or web browser application, such as a web browser application implemented using known web application frameworks such as Angular. System [100] may comprise process specification services [120] to implement functions such as the display, editing, receiving and storage of process specifications from the client application.

### Calculation Services

System [100] is further configured to carry out automatic calculations regarding process specifications, which substantially accelerates the biopharmaceutical development process. Said automatic calculations may be stored and executed by calculation services [130], which is typically implemented in a programming language such as Python. Upon receipt of any first parameter [43] dependent on a second parameter [44], system [100] is configured to automatically calculate the first parameter. The parameter dependency typically expresses a biopharmaceutical relationship, such as the liquid to solid ratio at the completion of a fermentation activity being depending on an inoculant concentration parameter during an inoculation step when other parameters such as temperature and time are met. As the number of steps in a process specification increases, the number of parameter dependencies will likely also increase, such that calculation services must automatically propagate changes to parameters throughout the process specification.

As will be described in more detail below, collaboration services [150] further ensure that users experience automatic calculations immediately after process specifications are edited on the process specification canvas. This means that a first user will automatically see the results of a change made by a second user, assuming both have the appropriate permissions. In other words, after receipt [211] of a process specification—such as a process specification made by a first user—and receipt [221] of an edited process specification—representing a change made by a second user—system [100] is configured to automatically re-calculate [231] parameters throughout the process specification, and moreover to store [241] and push [261] the edited process specification with calculated parameters to all users with the appropriate permissions.

System [100] is further configured to determine [251] which sites are capable of carrying out a step. Determination step [251] is illustrated in more detail in FIG. 13. The system [100] receives a site's equipment and equipment parameters [701] and receives facility fit logic rules [711] to define qualified equipment. For each activity, any facility fit logic rule [711] associated with the equipment and equipment parameters [701] associated with the activity is applied [721] to the activity parameters and the equipment parameters [701]. If the facility fit logic rule determines that the particular equipment is qualified [725], the comparison is repeated until each equipment at the site has qualified [726]. Every site for which all equipment [701] qualifies is added [731] to a set of qualified sites, and all qualified sites for a particular activity can be displayed [741]. It follows that when all steps of a process specification [49] can be carried out at a particular site, then a site qualified to carry out the process specification has been determined. Just as dependent parameters are automatically calculated, determination of the qualifying site or sites can also be carried out automatically.

System [100] is further configured to develop and transmit [281] the process specification for and to a qualified site, such that the process specification comprises all of the critical process parameters that are required to be maintained in order produce the biopharmaceutical recipe. Because users of system [100] are subject to identification verification, this transmittal [281] will be a secure transmittal when the client at the qualifying site is a user of system [100]. This is particularly important if the process specification has been approved for manufacture, thereby requiring compliance with Current Good Manufacturing Practice (CGMP) for drugs under FDA regulations. System [100] may be further configured to transform and transmit the process specification in a form readable to the site's manufacturing system, such as a Manufacturing Execution System (MES), Product Lifecycle Management (PLM) or Distributed Control System (DCS) software. This transformation is accomplished using an API, which can be implemented by enterprise application integration services [110]. Whereas process specifications were previously maintained in numbered versions in a variety of document forms, which required manual conversion to site-specific manufacturing systems, secure transmittal of process specifications to qualified sites reduces time spent creating site-appropriate process specifications.

Automatic calculation is particularly valuable as an enterprise proceeds through biopharmaceutical development. Research during a particular phase of development may focus on a larger set of parameters than the previous phase, but since it is likely that many parameter functions were determined in the previous phase, the automatic calculation of dependent parameters—and the sites qualified to manufacture them—would substantially accelerate the pace of development between one phase to the next.

### Collaboration Services

Furthermore, system [100] is configured using MQTT or other message-oriented middleware software infrastructure so that multiple users can access and edit a process specification simultaneously. This functionality is generally referred to as collaboration services [150], which comprises user permissions, user logs, workflow, and distributed messaging functionality.

Collaboration services [150] ensures that changes to a process specification are immediately be pushed [261] to users with adequate permissions for that process specification. Since calculation [231] is automatic, comprising an update to a process specification, all users accessing the process specification receive the stored update nearly instantaneously. This collaboration functionality also substantially accelerates the pace of development within a biopharmaceutical enterprise. Research and development of biopharmaceuticals is often performed by distinct groups working on separate process areas. For instance, a team working on cell growth is typically separate from a team that works on harvesting. In order to maintain control, including to comply with FDA CGMP requirements, recipe development is often performed sequentially, where upstream sequences of steps must be developed before handing off to a team working on a downstream set of activities. Because calculation is automatic, and moreover because the updated information is pushed to all users with adequate permissions (and logged), multiple users or user groups may view and work on a process specification simultaneously. Thus, system [100] eliminates the need for sequential development and facilitates distributed development, so that the pace of development is greatly accelerated overall.

User permissions allows the enterprise to maintain control over the process specification, including by ensuring that only users with appropriate permissions can access and/or edit process specifications or parts of process specifications, and by changing user permissions depending on the process specification's place in the overall biopharmaceutical development lifecycle. The degree to which a process specification must comply with institutional and agency regulations increases throughout the biopharmaceutical development lifecycle. For example, process specifications that have been approved for manufacturing are subject to tight control over user edits or even access. Storage [241] of each update may also comprise data regarding the user who made the update. As such, the system facilitates compliance with standards and regulations that mandate audit trails to prove the integrity of an electronic record, including FDA regulations over CGMP compliance. The system [100] is further configured to generate [271] a document showing the process specification with all of the details needed to support its review and validation. Where regulatory review of a particular process specification is required, the document can be configured to include the appropriate audit trails. Thus, the system [100] significantly reduces the time the enterprise requires to compile and consolidate product and process specification information into documents.

### Standardization Services

Because the system is capable of storing unlimited process specifications for any enterprise configuration, including unlimited versions of any given process specification, this data pool provides the opportunity for analysis and the ability to draw inferences across the pool of all process specifications. Using this data pool, the system can recommend baseline process specifications based on a specific product family, optimize parameters based on related variables within the process specification, identify portions of the process specification that may require more or less lab experimentation depending on degree of variation, and determine optimal resource productivity. Every process specification, along with its related metadata and notes tied to any data point, is stored in the process specification library. Therefore, from the outset of the long drug development process, the entire process specification is stored as a whole, enabling optimization services to provide insights based on the changes that were made during development.

In order to perform the foregoing analyses, enterprise configuration and process specification data is standardized and stored [802] in a process specification library. Any version of a process specification that is received [801] should be standardized and stored [802] in the standardized format. The data may come from the biopharmaceutical entity or organization itself, including sites within the organization, as well as related third parties such as Contract Manufacturing Organizations (CMOs), Contract Research Organizations (CROs), Contract Development Organizations (CDOs), or other pharmaceutical companies. Collected data may be de-identified, including by removing identifying product names, proprietary material names, site names, equipment configurations, and other sensitive information, and, where relevant, replacing them with standardized names. Pertinent metadata such as molecule type, recipe stage, recipe output quantity, number of revisions, comments regarding version changes, and other information relevant to classify the type of recipe is retained.

Process specification library input is standardized by first building a dictionary of synonyms to classify different names from disparate sources under the same term. Each step, parameter, process area, etc. in every enterprise configuration and process specification must have a standardized name, but each company and site may refer to the same concept differently. For example, different companies might use the terms “Temperature,” “Temp.,” “Deg. C,” or “Temperature Set Point” to describe the same aspect of a recipe.

Standardization may be performed using machine learning, statistics, or other means to derive a homogenized term for each item in the enterprise configurations and process specifications. The process should take into account enterprise configuration data as well as specific process specification data in order to place each term in its correct context within a process specification. For example, one company might use the parameter name “Production Bioreactor Temperature” to describe the temperature in the production bioreactor step of the process, whereas another company may use a more generic parameter name like “Temperature.” The standardized parameter name must be based on the step and process area combination with which the parameter is associated. Standardization identifies these different concepts across process specifications, compares each term to the existing library of synonyms, and either (a) determines which standardized term to translate a specific item into, or (b) identifies a new standardized term that needs to be created and if needed, flagged for human review.

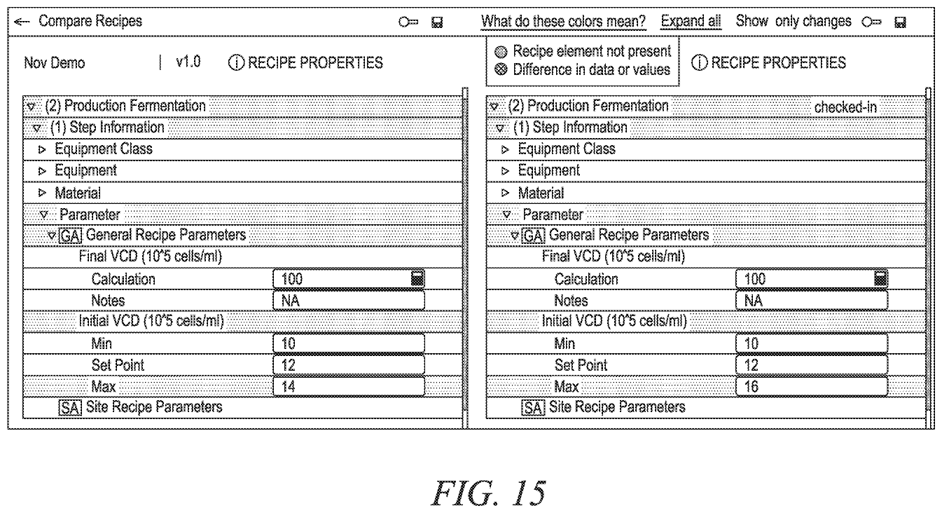

Standardization also enables the visual comparison of any two process specifications and the automatic identification of differences. For instance, in the example shown in FIG. 15, two process specifications are compared on the process specification canvas. Both process specifications have a step with the standardized name of “Production Fermentation,” and the parameters Initial and Final VCD. The comparison of the two process specifications reveals that they differ only in the Max value of the Initial VCD. The ability to compare any two process specifications can reduce the overall drug development time, for instance by comparing a target process specification to already existing process specifications in the process specification library.

### Optimization Services

By using the process specification library, various algorithms can be applied to analyze aspects of a process specification and/or provide a recommended process specification for a new molecule. Upon receipt [801] of a process specification, a set of similar process specifications can be determined [803] by finding similar process specifications in the library in the same product family (including type of cell line and type of drug), in the same lifecycle phase (e.g., pre-clinical, clinical phase 1, 2, or 3, or commercial), and/or with one or more matching steps and/or activities. The determination step [803] can be performed in a multi-step process, first by matching metadata such as product family, and optionally, lifecycle phase, followed by honing the set by matching steps. Matches are determined by comparing the terminology of the received process specification with the standardized term and their synonyms in the process specification library. Any matching step should take into account the process area of said step to its process area in the received process specification. The determination [803] of the analogous process specification can also be made by various algorithms that can compare and quantify the difference between datasets. The determination [803] may also be made in response to a requested product type or other data point, instead of a received [802] process specification. For instance, a user may request a particular cell line or molecule type in the pre-clinical phase of the development life cycle. The determination [803] may also be made in response to a set of experiment data input, insofar as such experiment data comprises one or more steps of a process specification that may be compared to process specifications in the library.

The set of similar process specifications can be returned as a listing with rankings or numerical quantification of the “percent match,” which the user may compare to other process specifications using a side-by-side comparison as discussed above. Alternatively, a synthesizing algorithm may be applied to the set of similar process specifications to return one recommended process specification. The synthesizing algorithm can be machine learning algorithms such as those configured to predict outcomes based on rules such as cubist rule-based decision tree models; neural networks such as monotonic multi-layer perceptron and related deep neural network models; and a combination of machine learning approaches such as genetic algorithms combined with fuzzy logic, or adaptive neuro-fuzzy inference models. For example, the recommended specification may include adding 2 bioreactor steps before the production bioreactor, using single-use bioreactors at a 2K liter scale, using two impellers on the agitator and multiple baffles in the bioreactor, and using anion-exchange chromatography and three washes in a particular process area. Once a recommended process specification is received, a user can choose to ‘propagate’ the differences from the recommended process specification to the current process specification.

In addition, the recommended process specification should identify and return [804] the most likely critical manufacturing parameters, such as critical process parameters (CPPs) or critical quality attributes (CQAs), so that a verifiable set of process instructions can be securely transferred to a manufacturing site. This data may be returned in the form of process instructions readable to manufacturing site automated execution systems. For example, the recommended specification may identify that in the Perfusion Bioreactor step, Temperature or pH are the most common CPPs/CQAs critical to determining the process quality or yield, and in the anion-exchange chromatography step, the parameter Flow Rate is critical to measure. CPPs/CQAs are the most critical sub-set of parameters that have been identified, often through years of experimentation and evidence collecting, to impact that specific molecule's manufacturing process to ensure the final product meets the required quality standards. As such, they are critical inputs to regulatory submissions and manufacturing monitoring processes. By identifying the CPPs/CQAs ahead of time, a company can greatly reduce their experimentation efforts to focus on a smaller subset of variables that are most likely to have an impact on their process. Furthermore, by having insights into the optimal values for each of those parameters at the outset (e.g., the Perfusion Bioreactor's Temperature Set Point should be 32, with an Upper Limit of 34 degrees, and a Lower Limit of 30 degrees), they can narrow the ranges they experiment on for each of those parameters, saving critical time and resources.

Using the process specification library, other types of insights may also be returned. Because data from every process specification revision made during the drug development process is stored, optimization services can derive insights from the types of changes that were made. For example, a step being revised many times compared to other steps in the set of similar process specifications, indicates the amount of effort required for that step. When accelerating time to market is critical, understanding which steps tend to go through more revisions is a valuable insight so companies can add additional resources to those steps earlier in a new drug's development process to avoid delays. By accessing metadata for equipment and similar process specifications across CMO, CRO, and CDO organizations, the system can also recommend which contract organization(s) may be a good fit for the process.

In response to a parameter optimization request [805], an optimization can be identified [807] by determining [806] a matching parameter in the set of similar process specifications and analyzing the steps and parameters that impacted that matching parameter across all versions/revisions of process specifications in the set of similar process specifications. For example, if the yield of the Production Bioreactor step is requested to be optimized [805], the changes/versions that led up to the final approval version of the set of similar process specifications can be analyzed to identify which parameters had the greatest impact on the production bioreactor yield. This analysis can be performed by various machine learning algorithms such as those configured to predict outcomes based on metaheuristic optimization techniques, such as particle swarm optimization. The most likely parameters to optimize yield may be, for example, not only the durations between feeds and the agitation rate, but also the potential values that might optimize the yield (e.g., feed the bioreactor every 3 days instead of every day, or adjust the agitation rate from 45 RPM to 55-65 RPM). The result of the parameter optimization request is returning the identification [807] of the set of parameters likely to optimize the requested parameter, as well as the range of values for each.

1. A manufacturing system for a biopharmaceutical product, the system comprising:
a first set of biopharmaceutical manufacturing equipment located at a first enterprise site at a first physical location, the first set of biopharmaceutical manufacturing equipment including multiple pieces of equipment each having one or more equipment parameters representing an operating capability of the piece of equipment;
a second set of biopharmaceutical manufacturing equipment located at a second enterprise site at a second physical location, the second set of biopharmaceutical manufacturing equipment including multiple pieces of equipment each having one or more equipment parameters representing an operating capability of the piece of equipment;
memory configured to store an enterprise configuration and a process specification,
the enterprise configuration including records of the one or more equipment parameters of the multiple pieces of equipment of the first set of biopharmaceutical manufacturing equipment and the one or more equipment parameters of the multiple pieces of equipment of the second set of biopharmaceutical manufacturing equipment, and
the process specification including a sequence of steps to manufacture the biopharmaceutical product and one or more step parameters corresponding to each of the steps, each of the one or more step parameters representing a physical attribute related to controlling a process of manufacturing the biopharmaceutical product; and
at least one processor configured to execute computer-executable instructions, wherein the instructions include
determining, based on the one or more step parameters for manufacturing the biopharmaceutical product and the one or more equipment parameters of the multiple pieces of equipment at each of the first enterprise site and the second enterprise site, whether at least one of the first enterprise site and the second enterprise site is capable of manufacturing the biopharmaceutical product,
in response to determining that at least one of the first enterprise site and the second enterprise site is capable of manufacturing the biopharmaceutical product, generating a set of instructions that are executable by the multiple pieces of equipment of the determined at least one of the first enterprise site and the second enterprise site, the set of instructions including one or more process control parameters that, when the set of instructions is executed by the multiple pieces of equipment of the determined at least one of the first enterprise site and the second enterprise site, control operation of the multiple pieces of equipment at the determined at least one of the first enterprise site and the second enterprise site,
transmitting the set of instructions to the determined at least one of the first enterprise site and the second enterprise site, and
operating the multiple pieces of equipment at the determined at least one of the first enterprise site and the second enterprise site according to the transmitted set of instructions, to manufacture the biopharmaceutical product.

2. The system of claim 1, wherein the multiple pieces of equipment include at least one of a tank, an agitator, a mixer, a column, a reaction vessel, a heat exchanger, and a cooling tower.

3. The system of claim 2, wherein:
a first portion of the multiple pieces of equipment of the first set of biopharmaceutical manufacturing equipment are arranged to work in sequence in a first manufacturing line; and
a second portion of the multiple pieces of equipment of the first set of biopharmaceutical manufacturing equipment are arranged to work in sequence in a second manufacturing line.

4. The system of claim 1, further comprising:
a first manufacturing execution system configured to communicate with the multiple pieces of equipment at the first enterprise site via a first network link; and
a second manufacturing execution system configured to communicate with the multiple pieces of equipment at the second enterprise site via a second network link.

5. The system of claim 4, wherein:
transmitting the set of instructions includes transmitting the set of instructions to the first manufacturing execution system or the second manufacturing execution system, corresponding to the determined at least one of the first enterprise site and the second enterprise site, and
operating the multiple pieces of equipment includes executing, by the corresponding first manufacturing execution system or second manufacturing execution system, the transmitted set of instructions.

6. The system of claim 1, wherein:
the enterprise configuration includes one or more facility fit logic rules each indicative of whether a corresponding one of the multiple pieces of equipment is qualified for at least one of the steps to manufacture the biopharmaceutical product based on one or more operating parameters of the corresponding one of the multiple pieces of equipment and one or more step parameters corresponding to the at least one step; and
determining whether at least one of the first enterprise site and the second enterprise site is capable of manufacturing the biopharmaceutical product includes applying at least one of the one or more facility fit logic rules to the multiple pieces of equipment of the first enterprise site and the second enterprise site.

7. The system of claim 1, wherein each of the sequence of steps includes at least one parameter dependency, and the instructions further include automatically calculating parameter dependencies of a site-specific sequence of steps corresponding to the determined at least one of the first enterprise site and the second enterprise site, to generate a site-specific process specification.

8. The system of claim 7, wherein the instructions further include:
receiving an updated process specification including a change to at least one of the sequence of steps; and
automatically recalculating the at least one parameter dependency to generate an updated site-specific process specification.

9. The system of claim 7, wherein the instructions include:
displaying a set of qualified enterprise sites to permitted users;
receiving an enterprise site selection from at least one of the permitted users;
determining a site-specific process specification for the selected enterprise site;
generating a regulatory process transfer document including the determined site-specific process specification and for the selected enterprise site and a user identification; and
automatically controlling access to the regulatory process transfer document based on the user identification.

10. The system of claim 1, further comprising a visual interface including a process specification canvas configured to display the process specification and receive edit inputs to the process specification.

11. The system of claim 1, wherein the memory is configured to store a process specification library, the process specification including a set of standardized process specifications having standardized terms and synonyms for each step, parameter and process area, and the instructions further include:
determining at least one similar standardized process specification from the process specification library which matches the process specification comprising the sequence of steps to manufacture the biopharmaceutical product.

12. The system of claim 11, wherein the instructions further include determining a set of critical manufacturing parameters based on the determined at least one similar standardized process specification from the process specification library.

13. The system of claim 11, wherein the instructions further include:
receiving a parameter optimization request;
identifying a matched parameter in the determined at least one similar standardized process specification from the process specification library; and
determining a set of parameters and corresponding values that result in a greatest change in the matched parameter, by comparing version changes across all versions of all process specifications in the determined at least one similar standardized process specification from the process specification library.

14. A method for automated manufacturing of a biopharmaceutical product, the method comprising:
obtaining an enterprise configuration including records of one or more equipment parameters of multiple pieces of equipment of a first set of biopharmaceutical manufacturing equipment located at a first enterprise site at a first physical location, and one or more equipment parameters of multiple pieces of equipment of a second set of biopharmaceutical manufacturing equipment located at a second enterprise site at a second physical location;
obtaining a process specification including a sequence of steps to manufacture the biopharmaceutical product and one or more step parameters corresponding to each of the steps, each of the one or more step parameters representing a physical attribute related to controlling a process of manufacturing the biopharmaceutical product;
determining, based on the one or more step parameters for manufacturing the biopharmaceutical product and the one or more equipment parameters of the multiple pieces of equipment at each of the first enterprise site and the second enterprise site, whether at least one of the first enterprise site and the second enterprise site is capable of manufacturing the biopharmaceutical product;
in response to determining that at least one of the first enterprise site and the second enterprise site is capable of manufacturing the biopharmaceutical product, generating a set of instructions that are executable by the multiple pieces of equipment of the determined at least one of the first enterprise site and the second enterprise site, the set of instructions including one or more process control parameters that, when the set of instructions is executed by the multiple pieces of equipment of the determined at least one of the first enterprise site and the second enterprise site, control operation of the multiple pieces of equipment at the determined at least one of the first enterprise site and the second enterprise site;
transmitting the set of instructions to the determined at least one of the first enterprise site and the second enterprise site; and
operating the multiple pieces of equipment at the determined at least one of the first enterprise site and the second enterprise site according to the transmitted set of instructions, to manufacture the biopharmaceutical product.

15. The method of claim 14, wherein the multiple pieces of equipment include at least one of a tank, an agitator, a mixer, a column, a reaction vessel, a heat exchanger, and a cooling tower.

16. The method of claim 14, wherein:
the enterprise configuration includes one or more facility fit logic rules each indicative of whether a corresponding one of the multiple pieces of equipment is qualified for at least one of the steps to manufacture the biopharmaceutical product based on one or more operating parameters of the corresponding one of the multiple pieces of equipment and one or more step parameters corresponding to the at least one step; and
determining whether at least one of the first enterprise site and the second enterprise site is capable of manufacturing the biopharmaceutical product includes applying at least one of the one or more facility fit logic rules to the multiple pieces of equipment of the first enterprise site and the second enterprise site.

17. The method of claim 14, wherein each of the sequence of steps includes at least one parameter dependency, and the instructions further include automatically calculating parameter dependencies of a site-specific sequence of steps corresponding to the determined at least one of the first enterprise site and the second enterprise site, to generate a site-specific process specification.

18. The method of claim 14, further comprising:
accessing a process specification library, the process specification including a set of standardized process specifications having standardized terms and synonyms for each step, parameter and process area, and the instructions further include:
determining at least one similar standardized process specification from the process specification library which matches the process specification comprising the sequence of steps to manufacture the biopharmaceutical product.

19. The method of claim 18, wherein the instructions further include:
receiving a parameter optimization request;
identifying a matched parameter in the determined at least one similar standardized process specification from the process specification library; and
determining a set of parameters and corresponding values that result in a greatest change in the matched parameter, by comparing version changes across all versions of all process specifications in the determined at least one similar standardized process specification from the process specification library.

20. A computer program product embodied on a non-transitory computer readable-medium, the computer program product comprising instructions to cause a processor to:
receive and store in a memory, an enterprise configuration that includes records of:
a plurality of physical locations at which an enterprise manufactures one or more biopharmaceutical products;
one or more pieces of equipment corresponding to each of the physical locations; and
one or more equipment parameters corresponding to each of the pieces of equipment, wherein each equipment parameter represents a capability the piece of equipment corresponding to the parameter;
receive and store in a memory, a process specification that includes records, for manufacturing each of the one or more biopharmaceutical products, of:
a sequence of steps to manufacture the biopharmaceutical product; and
one or more step parameters corresponding to each of the steps, wherein each of the one or more step parameters represents a physical attribute related to controlling the process of manufacturing the biopharmaceutical product;
determine, based on the step parameters for manufacturing a particular biopharmaceutical product and the equipment parameters corresponding to each of the pieces of equipment for a particular physical location, whether the particular physical location is capable of manufacturing the particular biopharmaceutical product;
generate, when it is determined that the particular physical location is capable of manufacturing the particular biopharmaceutical product and based on the process specification for the particular biopharmaceutical product, a set of instructions that are executable by a manufacturing execution system of the particular physical location to manufacture the particular biopharmaceutical product, wherein the set of instructions includes one or more process control parameters that, when the set of instructions is executed by the manufacturing execution system, control operation of the one or more pieces of equipment at the particular physical location; and
transmit the set of instructions to the particular physical location.
